(19) United States
(12) Patent Application Publication
(10) Pub. No.: US 2013/0166006 A1
(43) Pub. Date: Jun. 27, 2013

(54) Neuromodulation Systems and Methods for Treating Epilepsy

(71) Applicant: Michael S. Williams, Santa Rosa, CA (US)

(72) Inventor: Michael S. Williams, Santa Rosa, CA (US)

(21) Appl. No.: 13/530,093

(22) Filed: Jun. 21, 2012

Related U.S. Application Data
(60) Provisional application No. 61/499,180, filed on Jun. 21, 2011.
(60) Provisional application No. 61/500,031, filed on Jun. 22, 2011.

(51) Int. Cl. A61N 1/05

(52) U.S. Cl. CPC A61N 1/0551
USPC 607/116

(57) ABSTRACT

A neuromodulation system for treating epilepsy provides therapeutic elements for modulating nerve activity to prevent or diminish (e.g. through reduced intensity or shortened duration) epileptic seizures. The therapeutic elements may be positioned in the vasculature of the patient and are energized to modulate nerve fibers positioned outside the vascular walls. Electrode positions may include the maxillary vein, inferior alveolar vein, lingual vein, retromandibular or facial vein, or the emissary vein of the foramen ovale. Target nerves include the mandibular branch (V3) of the trigeminal nerve, or a branch of the mandibular branch (e.g. the inferior alveolar nerve).

This application claims the benefit of U.S. Provisional Application No. 61/499,180, filed Jun. 21, 2011, and U.S. Provisional Application No. 61/500,031, filed Jun. 22, 2011, each of which is incorporated herein by reference.

### TECHNICAL FIELD OF THE INVENTION

The present application generally relates to systems and methods for treating epilepsy through neuromodulation.

### BACKGROUND

Applicant's prior Application Publication No. U.S. 2007/0255379 ('379 application), which is incorporated herein by reference, discloses a neurostimulation device and associated methods for stimulating nervous system targets. In disclosed embodiments, neurostimulation is achieved using an electrode positioned in the vasculature in proximity to a target nervous system target such as, for example, a nerve. In use of such a system, stimulation can be targeted to one or more nerves to enhance, augment, inhibit or block signaling of efferent, afferent and/or interneuronal nerve cells, with any combination of these effects being within the scope of the disclosure. Stimulus can be directed to a mixed nerve containing both afferent and efferent nerve cells to produce one effect (e.g. enhance, inhibit or block signaling) on one type of nerve cell (i.e. the afferent or efferent nerve cells), and to produce the same or a different effect (e.g. enhance, inhibit, block, or yield a neutral effect) on the other type of nerve cell. Alternatively, stimulation can be delivered to one or more separate afferent nerves, efferent or interneuronal nerves using the same or different electrodes/fluid conduits to trigger one of these effects (e.g. enhance signaling, inhibit signaling, block signaling, or have a neutral or any combination of the effects).

The '379 application discloses that the system may be suitable for use in treating epilepsy. The present application describes further embodiments suitable for epilepsy treatment.

### BRIEF DESCRIPTION OF THE DRAWINGS

FIG. 1 is a drawing of a human head schematically illustrating the positioning of veins and nerves relevant to use of the disclosed system;

FIG. 2 schematically illustrates positioning of a fully intravascular system for epilepsy treatment.

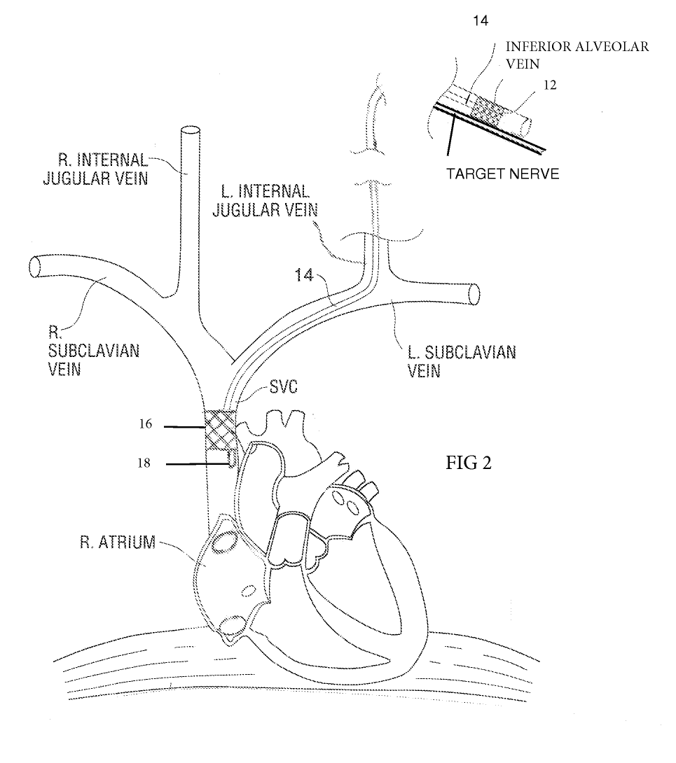

### DETAILED DESCRIPTION

A neuromodulation system for treating epilepsy provides therapeutic elements for modulating nerve activity to prevent or diminish (e.g. through reduced intensity or shortened duration) epileptic seizures. In preferred embodiments, the therapeutic elements are positioned in the vasculature of the patient and are energized to modulate nerve fibers positioned outside the vascular walls. The therapeutic elements may be delivered to a target site within the vasculature using methods described in the '379 applications. In some embodiments, access to the target site is achieved through the right or left femoral vein, the subclavian or brachiocephalic veins, or the cephalic, basilica, or median cubital veins). Modulation may be carried out to activate and/or inhibit activation of target nerve fibers. In the disclosed system, the therapeutic elements are described as electrodes, although it is contemplated that other forms of therapeutic elements (including, but not limited to, ultrasound, thermal, or optical elements) may instead be used.

The therapeutic elements may be positioned on leads extending through the vasculature and coupled to an implanted pulse generator. Intravascular pulse generators and associated components are shown and described in the '379 application, and U.S. Pat. Nos. 7,082,336, U.S. 7,529,589, US 7,363,082, and U.S. 7,840,282, each of which is incorporated by reference. The pulse generator may be an intravascular pulse generator positioned in a blood vessel such as the superior vena cava, inferior vena cava, left subclavian vein, or other blood vessel. Alternatively, the pulse generator may be positioned subcutaneously, with the leads extending from the pulse generator into the vasculature. In other embodiments, the intravascular therapeutic elements may be physically independent from the pulse generator, in which case the therapeutic elements would be wirelessly powered by the pulse generator, such as through inductive coupling, or vibrational or ultrasound transmission. In such cases, the pulse generator may be subcutaneous, intravascular, or extracorporeal. In one such example, an inductively powered electrode is positioned within the target blood vessel and wirelessly energized by a subcutaneous pulse generator to direct neuromodulation therapy to the target nerve.

The nerve fibers may be modulated from a single therapeutic element or multiple elements, such as select electrodes within an electrode array. The blood vessel and the target position of the therapeutic elements within a chosen vessel is selected based on the vessel's anatomic location relative to the target fiber so as to position the therapeutic element in close proximity to the target fiber while minimizing collateral effects. For controlling epilepsy, the target nerve fiber(s) and/or the target vessel may be located on the patient's head. In some embodiments, neuromodulation is targeted to a branch of the trigeminal nerve. In some embodiments, the targeted branch is the mandibular branch (V3) of the trigeminal nerve, or a branch of the mandibular branch (e.g. the inferior alveolar nerve). Suitable electrode sites for achieving stimulus of these nerve structures include venous vessels accessible via the internal or external jugular vein. Examples include but are not limited to the maxillary vein, inferior alveolar vein, lingual vein, retromandibular or facial vein, or the emissary vein of the foramen ovale. In each case, the electrode is positioned within a portion of the vessel that is in proximity to the target nerve, such that activation of the electrode(s) directs the therapy to the target nerve(s). For example, in one embodiment one or more electrodes are positioned in the inferior alveolar vein or the maxillary vein at a location in proximity to the inferior alveolar nerve (see attached Figure). Access to these veins may be achieved by passing the electrode through internal jugular vein, through the common trunk for the facial, retromandibular and lingual veins, through the retromandibular vein to the maxillary vein, and (where desired) into the inferior alveolar vein, which runs in parallel to the inferior alveolar nerve.

If necessary to avoid migration of the therapeutic element within the vasculature, the therapeutic element may be retained within the target vessel using an expandable anchor, including but not limited to an anchor of the type disclosed in the '379 application and those disclosed in the other patents and applications incorporated herein. If an intravascular pulse generator is utilized, similar anchors may be used to retain the pulse generator within the vasculature.

Anchor and electrode arrangements for delivering transvenous electrical therapy to target nerves are known in the art. Some such arrangements, which bias the electrodes against the vascular wall to optimize transmission of therapeutic energy through the wall to the target vessel, are described in the '379 application and PCT Publication WO/2012/030393, which are incorporated herein by reference.

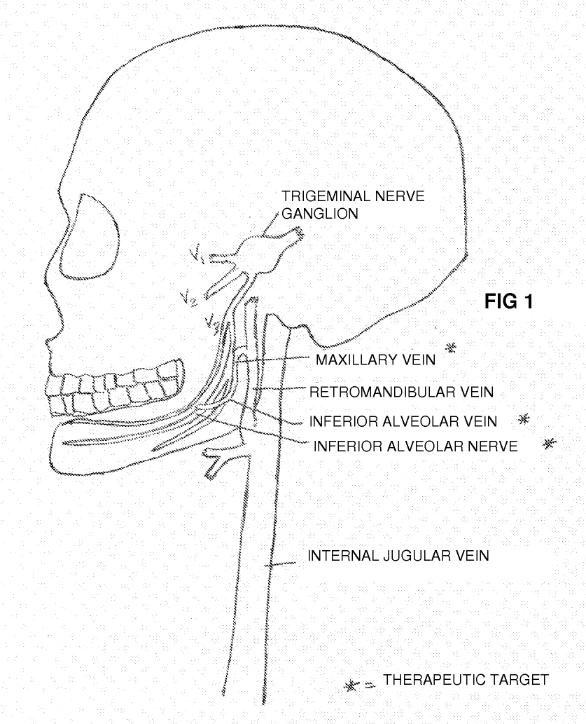

FIG. 1 illustrates one such anchor/electrode 12 disposed within an inferior alveolar vein positioned to deliver therapy transvenously to the inferior alveolar vein. The anchor/electrode 12 is carried by a lead 14 coupled to a pulse generator 18. An expandable anchor retains the pulse generator 18 within the SVC.

The therapeutic element may be part of a system that senses physiological activity and determines the onset of an epileptic seizure or other changes indicative that a seizure is likely, and that delivers the neuromodulation therapy in response to the detected onset or change. In other embodiments, the patient may have an external controller in wireless communication with the therapeutic implant, allowing the patient to initiate neuromodulation when s/he senses the onset of an epileptic seizure.

Other embodiments may be employed which do not use an intravascular therapeutic element. For example, the therapeutic element may be attached to the patient's skin—such as on the jaw or another part of the face. In one such embodiment, a patch is mounted to the patient's skin using an adhesive, with the therapeutic element positioned to deliver neuromodulation therapy through the skin to target nerve fiber(s). Such a therapeutic element may comprise electrode(s) positioned in contact with the skin. Electrodes suitable for this purpose include but are not limited to regions of conductive material printed or otherwise deposited onto a flexible substrate. Such a patch may be disposable such that, for example, the patient replaces a used patch with a new replacement patch on a daily or weekly basis, or once the used patch has delivered neuromodulation therapy a predetermined number of times.

In one embodiment, the patch contains all of the elements needed to energize the electrodes, such as a battery and the associated electronics. An external device might be positioned in wireless communication with the patch for use in programming the patch electrodes and/or controlling delivery of therapy. In an alternative embodiment the patch may be provided without a battery or other power supply for energizing the electrodes, in which case energy may be coupled (e.g. inductively or by other means known to those skilled in the art) to the electronics housed within the patch using a second external device. The coupled energy energizes the electrodes for delivering neuromodulation therapy. In this latter embodiment the external device might also be used as a wireless programmer or control unit for the patch. In embodiments using an external device, the external device may be configured to be externally worn on the body (e.g. coupled to or hung on the ear as is done with a hearing aid) or integrated into a garment such as a scarf or collar.

In another embodiment, the therapeutic element may be a subcutaneous device positioned beneath the skin through a small incision or injected beneath the skin using a needle. The subcutaneous device may take many forms, including but not limited to a flexible patch-like device having characteristics similar to the patch described above, or an injectable capsule. The subcutaneous device may be self-controlling and self-powered using its own battery and electronics, or it may work in combination with an external device as disclosed in the preceding paragraph.

All prior patents and applications referred to herein, including for purposes of priority, are incorporated by reference for all purposes.

It should be recognized that a number of variations of the above-identified embodiments will be obvious to one of ordinary skill in the art in view of the foregoing description. Moreover, it is contemplated that aspects of the various disclosed embodiments may be combined to produce further embodiments. Accordingly, the invention is not to be limited by those specific embodiments and methods of the present invention shown and described herein. Rather, the scope of the invention is to be defined by the following claims and their equivalents.

1. A method for treating epilepsy, the method comprising:
positioning a therapeutic element in a blood vessel on a head of a patient; and
transvascularly delivering therapeutic energy from the therapeutic element to a nerve proximate to the blood vessel such that delivery of the therapeutic energy prevents or diminishes epileptic seizure activity of the patient.

2. The method of claim 1, wherein the nerve is the mandibular branch (V3) of the trigeminal nerve.

3. The method of claim 1, wherein the nerve is a branch of the mandibular branch of the trigeminal nerve.

4. The method of claim 1, wherein the nerve is the inferior alveolar nerve.

5. The method of claim 1, wherein the blood vessel is selected from the group consisting of maxillary vein, inferior alveolar vein, lingual vein, retromandibular or facial vein, or the emissary vein of the foramen ovale.

6. A system for treating epilepsy, comprising:
a therapy element adapted for positioning within a blood vessel on a head of a patient; and
a stimulator configured to energize the therapy element within the blood vessel to deliver therapy to a nerve fiber disposed external to the blood vessel such that delivery of the therapy prevents or diminishes epileptic seizure activity of the patient.
